(19) United States
(12) Patent Application Publication
(10) Pub. No.: US 2022/0323573 A1
(43) Pub. Date: Oct. 13, 2022

(54) METHODS FOR TREATING COVID-19 BASED ON INDIVIDUAL GENOMIC PROFILES

(71) Applicant: University of South Florida, Tampa, FL (US)

(72) Inventors: Jose D. Herazo-Maya, Tampa, FL (US); Brenda M. Juan-Guardela, Tampa, FL (US); Gaetane Michaud, Tampa, FL (US)

(21) Appl. No.: 17/713,382

(22) Filed: Apr. 5, 2022

Related U.S. Application Data
(60) Provisional application No. 63/170,634, filed on Apr. 5, 2021.

(51) Int. Cl. A61K 39/215; A61K 31/573; A61K 31/7064; A61K 31/427; A61K 38/06; A61K 39/42; A61P 31/14; A61K 31/675

(52) U.S. Cl. CPC A61K 39/215; A61K 31/573; A61K 31/7064; A61K 31/427; A61K 38/06; A61K 39/42; A61P 31/14; A61K 31/675

(57) ABSTRACT

Disclosed are gene risk profiles that predict outcomes in COVID-19. Single-cell analysis reveals immune responses associated with COVID-19 mortality.

### CROSS-REFERENCE TO RELATED APPLICATIONS

This application claims priority to U.S. Provisional Application No. 63/170,634, filed Apr. 5, 2021, which is incorporated by reference in its entirety for all purposes.

### FIELD

The present disclosure relates to gene expression profiles and methods for treating COVID-19.

### BACKGROUND

COVID-19 is associated with increased mortality and poor outcomes in individuals at risk. Unfortunately, it is difficult to predict which patients will deteriorate, require ICU admission, mechanical ventilation and die from the disease. While patients who receive invasive mechanical ventilation are more likely to be males, obese, and to have elevated values of liver-function tests and inflammatory markers (ferritin, d-dimer, C-reactive protein, and procalcitonin), reliable, peripheral blood biomarkers of COVID-19 severity and mortality are still necessary to better triage patients and improve utilization of resources. Pulmonary fibrosis has overlapping risk factors for severe COVID-19 and has been reported as a recognized sequelae of COVID-19 infection (Spagnolo 2020). A peripheral blood gene signature has been shown to predict mortality in Idiopathic Pulmonary Fibrosis (IPF).

What is needed in the art is a method of distinguishing which patients are more likely to develop severe COVID-19, so that those patients can be treated accordingly.

### SUMMARY

Disclosed herein is a method of treating a subject with severe COVID-19 or at risk of developing severe COVID-19, the method comprising: obtaining a sample from the subject; measuring expression level of at least two of the following genes from the subject: PLBD1, TPST1, MCEMP1, IL1R2, HP, FLT3 and S100A12; determining a score for the measured expression level of the at least two genes, using these scores to determine a severity signature profile for the subject; determining that the severity signature profile is greater than a gene-specific standard; and treating the subject for severe COVID-19, or treating the patient to prevent severe COVID-19.

Also disclosed herein is a method of treating a subject with severe COVID-19 or at risk of developing severe COVID-19, the method comprising: obtaining a sample from the subject; measuring expression level of at least one of the following genes from the subject: PLBD1, TPST1, MCEMP1, IL1R2, HP, FLT3 and S100A12; measuring expression level of at least one of the following genes from the subject: LCK, CAMK2D, NUP43, SLAMF7, LRRC39, ICOS, CD47, LBH, SH2D1A, CNOT6L, METTL8, ETS1, P2RY10, TRAT1, BTN3A1, LARP4, TC2N, GPR183, MORC4, STAT4, LPAR6, CPED1, DOCK10, ARHGAP5, HLA-DPA1, BIRC3, GPR174, CD28, UTRN, CD2, HLA-DPB1, ARL4C, BTN3A3, CXCR6, DYNC2LI1, BTN3A2, ITK, CD96, GBP4, S1PR1, NAP1L2, KLF12, IL7R); determining a score for the measured gene expression level of the at least two genes from the list of 7 genes, the list of 43 genes, or a combination of one or more of both; using the score to obtain a severity signature profile; determining that the severity signature profile is greater than a gene-specific standard; treating the subject for severe COVID-19 or to prevent severe COVID-19.

### BRIEF DESCRIPTION OF DRAWINGS

The accompanying figures, which are incorporated in and constitute a part of this specification, illustrate several aspects described below.

FIGS. 1A-1B show 50-gene risk profiles are predictive of COVID-19 outcomes. Clustering of COVID-19 subjects based on 50-gene risk profiles (High Vs. Low) determined by SAMS in discovery (A) and validation cohorts (B). Every column represents a subject and every row represents a gene. Log-based two color scale is shown next to heatmaps; red denotes increase expression over the median of samples and green, decrease. S=Severe COVID-19 disease. M=Mild COVID-19.

FIGS. 2A-2D show 50-gene risk profile cutoffs predictive of COVID-19 severity are predictive of poor IPF outcomes. (A) Clustering of IPF-University of Chicago and (B) IPF-Imperial College London cohort based on genomic risk profiles (High Vs Low) derived from the COVID-19-Discovery cohort. Every row represents a gene and every column a patient. Color scale is shown adjacent to heat maps in log-based two scale; red denotes increase over the median of samples and green, decrease (C) Mortality differs between 50-gene risk profiles in the IPF-University of Chicago and IPF-Imperial College London cohort.

FIGS. 3A-3B show gene expression analysis of 50-genes in single cells from the COVID-19-Single cell cohort demonstrating differences in risk profiles and cell proportions. (A) Heatmap shows cell types with 50-gene, low-risk versus high-risk expression profiles. Every column represents a single cell and every row represents a gene. Log-based two color scale is shown next to heatmap; red denotes increase expression over the median of samples and green, decrease. (B) Proportion of 50-gene expressing cells in high Vs low risk profiles. Y axis represents cell types and X axis represents cell proportions. B: B Cell, CD4m T: Memory CD4 T Cell, CD4n T: Naive CD4 T Cell, CD8m T: Memory CD8 T Cell, DC: Conventional Dendritic Cell, gd T: Gamma Delta T cells, IFN-stim CD4 T: Interferon-stimulated CD4 T cell, IgA PB: IgA (Immunoglobulin-A) Plasmablast, IgG PB: IgG (Immunoglobulin-G) Plasmablast, IgM PB: IgM (Immunoglobulin-M) Plasmablast, NK: Natural Killer Cell, pDC: Plasmacytoid Dendritic Cell, Myeloid cells, SC & Eosinophil: Stem Cells and Eosinophil.

FIGS. 4A-4C show up scores derived from seven increased genes of the 50-gene signature are predictive of mortality in COVID-19. To determine whether seven over-expressed genes of the 50-gene signature could be predictive of COVID-19 mortality, the same SAMS Up score cutoffs derived from the COVID-19-Discovery cohort (Up Score >0.41) were used, and applied them to the COVID-19-Validation cohort. The results demonstrate that an up score of >0.41 can distinguish low and high-risk subgroups of COVID-19 patients (FIG. 4A) based on increased expression of PLBD1, TPST1, MCEMP1, IL1R2, HP, FLT3 and S100A12. Every column represents a subject and every row represents a gene. Log-based two-color scale is shown next to the heatmap. Kaplan Meier curves demonstrated that none of the patients in the 7-gene, low risk group died from COVID-19 whereas all of the patients who died during the study period had a 7-gene, high-risk profile (P=0.042) (FIG. 4B). The prediction of in-hospital mortality was superior using 7-Gene Up scores compared to IL-6, CCL2 and OPN plasma levels (FIG. 4C). Taken together, these results show that a 7-gene signature can be sufficient to predict COVID-19 mortality compared to the entire 50-gene signature.

FIG. 5 shows 7-Gene Up score is associated with treatment response in COVID-19. X axis represents the 7-Gene Up score measured by qRT-PCR. The Y axis represents the days of hospitalization. 7-Gene Up score declined during hospitalization in COVID-19 treated patients who survived. 7-Gene Up score increased in treated patients who died during hospitalization.

FIGS. 6A-6C show S100A12 plasma levels appear to be informative of treatment response in COVID-19. Plasma levels of S100A12 in patients from the USF/TGH cohort are significantly correlated with their qRT-PCR transcript levels (FIG. 6A). Plasma levels of S100A12 are significantly higher in patients with a 7-Gene, high versus low risk profile (FIG. 6B) and seem to be associated with treatment response in COVID-19 (FIG. 6C). Decreased S100A12 plasma levels were seen in treated survivors during hospitalization. Increased S100A12 plasma levels were seen during hospitalization in patients who died despite COVID-19 treatments.

FIGS. 7A-7B. cDNA quality control preceding library construction for single-cell RNA sequencing experiments using Chromium 10×. Migration profile (FIG. 7A) and electropherogram (FIG. 7B) of the three cDNA samples measured by an Agilent High Sensitivity D1000 ScreenTape assay kit to determine cDNA amplicon size.

### DETAILED DESCRIPTION

### General Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduce symptoms” means reducing symptoms relative to a standard or a control.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, such as COVID-19, wherein the subject may or may not exhibit heightened susceptibility to the disorder or event.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

“Algorithm” is a set of rules for describing a biological condition. The rule set may be defined exclusively algebraically but may also include alternative or multiple decision points requiring domain-specific knowledge, expert interpretation or other clinical indicators.

A “Gene-Specific Standard” is a set of values associated with constituents of a Gene Expression Panel resulting from evaluation of a biological sample (or population or set of samples) under a desired biological condition that is used for mathematically normative purposes. The desired biological condition may be, for example, the condition of a subject (or population or set of subjects) before exposure to an agent or in the presence of an untreated disease or in the absence of a disease. Alternatively, or in addition, the desired biological condition may be health of a subject or a population or set of subjects. Alternatively, or in addition, the desired biological condition may be that associated with a population or set of subjects selected on the basis of at least one of age group, gender, ethnicity, geographic location, nutritional history, medical condition, clinical indicator, medication, physical activity, body mass, and environmental exposure.

A “set” or “population” of samples or subjects refers to a defined or selected group of samples or subjects wherein there is an underlying commonality or relationship between the members included in the set or population of samples or subjects.

“Body fluid” of a subject includes blood, urine, spinal fluid, lymph, mucosal secretions, prostatic fluid, semen, haemolymph or any other body fluid known in the art for a subject.

“Calibrated profile data set” is a function of a member of a first profile data set and a corresponding member of a baseline profile data set for a given constituent in a panel.

A “clinical indicator” is any physiological datum used alone or in conjunction with other data in evaluating the physiological condition of a collection of cells or of an organism. This term includes pre-clinical indicators.

A “composition” includes a chemical compound, a nutriceutical, a pharmaceutical, a homeopathic formulation, an allopathic formulation, a naturopathic formulation, a combination of compounds, a toxin, a food, a food supplement, a mineral, or a complex mixture of substances, in any physical state or in a combination of physical states.

To “derive” a profile data set from a sample includes determining a set of values associated with constituents of a Gene Expression Panel either (i) by direct measurement of such constituents in a biological sample or (ii) by measurement of such constituents in a second biological sample that has been exposed to the original sample or to matter derived from the original sample.

A “Gene Expression Panel” is an experimentally verified set of constituents, each constituent being a distinct expressed product of a gene, whether RNA or protein, wherein constituents of the set are selected so that their measurement provides a measurement of a targeted biological condition.

A “Gene Expression Profile” or “Severity Signature Profile” is a set of values associated with constituents of a Gene Expression Panel resulting from evaluation of a biological sample (or population or set of samples).

“Index” is an arithmetically or mathematically derived numerical characteristic developed for aid in simplifying or disclosing or informing the analysis of more complex quantitative information. A disease or population index may be determined by the application of a specific algorithm to a plurality of subjects or samples with a common biological condition.

A “normative” condition of a subject to whom a composition is to be administered means the condition of a subject before administration, even if the subject happens to be suffering from a disease.

A “panel” of genes is a set of genes including at least two constituents.

A “sample” from a subject may include a single cell or multiple cells or fragments of cells or an aliquot of body fluid, taken from the subject, by means including venipuncture, excretion, ejaculation, massage, biopsy, needle aspirate, lavage sample, scraping, surgical incision or intervention or other means known in the art. The sample can be sputum, blood, or mucous, such as obtained from a nasal swab.

### Methods of Diagnosing and Treating COVID-19 Patients

Peripheral blood gene expression datasets from two COVID-19 cohorts (Discovery and Validation) and two Idiopathic Pulmonary Fibrosis (IPF) cohorts were studied. When Scoring Algorithm of Molecular Subphenotypes (SAMS) was applied in all cohorts, high and low-risk subgroups were identified based on the expression of a set of 7 genes, and a larger set of 50 genes, which includes the 7 genes of the first set. In the first COVID-19-cohort (Discovery), 50-gene risk profiles distinguished mild from severe disease. SAMS cutoffs derived from this cohort were significantly predictive of poor outcomes in a COVID-19-Validation cohort and in two IPF cohorts. Single-cell analysis of 50-gene risk profiles was performed in COVID-19 subjects and single cells and identified the key cells responsible for the gene expression changes. Further detail can be found in Example 1.

Therefore, disclosed herein is a method of treating a subject with severe COVID-19 or at risk of developing severe COVID-19, the method comprising: obtaining a sample from the subject; measuring expression level of at least two of the following genes from the subject: PLBD1, TPST1, MCEMP1, IL1R2, HP, FLT3 and S100A12; determining a score for the measured expression level of the at least two genes, using these scores to determine a severity signature profile for the subject; determining that the severity signature profile is greater than a gene-specific standard; and treating the subject for severe COVID-19, or treating the patient to prevent severe COVID-19.

Also disclosed herein is a method of treating a subject with severe COVID-19 or at risk of developing severe COVID-19, the method comprising: obtaining a sample from the subject; measuring expression level of at least one of the following genes from the subject: PLBD1, TPST1, MCEMP1, IL1R2, HP, FLT3 and S100A12; measuring expression level of at least one of the following genes from the subject: LCK, CAMK2D, NUP43, SLAMF7, LRRC39, ICOS, CD47, LBH, SH2D1A, CNOT6L, METTL8, ETS1, P2RY10, TRAT1, BTN3A1, LARP4, TC2N, GPR183, MORC4, STAT4, LPAR6, CPED1, DOCK10, ARHGAP5, HLA-DPA1, BIRC3, GPR174, CD28, UTRN, CD2, HLA-DPB1, ARL4C, BTN3A3, CXCR6, DYNC2LI1, BTN3A2, ITK, CD96, GBP4, S1PR1, NAP1L2, KLF12, IL7R); determining a score for the measured gene expression level of the at least two genes from the list of 7 genes, the list of 43 genes, or a combination of one or more of both; using the score to obtain a severity signature profile; determining that the severity signature profile is greater than a gene-specific standard; treating the subject for severe COVID-19 or to prevent severe COVID-19.

By “severe COVID-19” is meant one or more of the following conditions are met (Zheng et al. Patterns. Vol. 1, Issue 6, 100092, Sep. 11, 2020):
(1) Respiratory distress (respiratory rate ≥30 breaths/minute);
(2) Peripheral capillary oxygen saturation (SpO₂)≤93%;
(3) Arterial partial pressure of oxygen (PaO₂)/fraction of inspired oxygen (FiO₂)≤300 mmHg;
(4) Pulmonary lesion progression exceeds 50% in 24-48 hours;
(5) Respiratory failure that requires mechanical ventilation;
(6) Shock; and/or
(7) Organ failure that requires to be managed in intensive care unit.

Severe COVID-19 can also mean that the subject reports one or more of the following symptoms: constant trouble breathing, persistent chest pain or pressure, confusion, trouble staying awake, blue lips or face.

It is noted that “severe COVID-19” is distinguished from mild or moderate COVID-19 based on the above metrics or reported symptoms. It is important to note that the subject may not show any signs or symptoms of COVID-19 when the sample is obtained from the subject. Alternatively, the subject may show mild or moderate symptoms of COVID-19, meaning that they do not show any of the signs and symptoms discussed above in connection with severe COVID-19, or they show less than all, or less than 6, 5, 4, 3, or 2 of the signs and symptoms. In the case where the subject shows no symptoms, or moderate or mild symptoms of COVID-19, the subject would not have been treated for severe COVID-19 but for the severity signature profile. In other words, the subject would not have been treated at all, or would have been treated for mild or moderate symptoms, but for the severity signature profile obtained using the method described herein.

In one aspect, the subject who is determined to have a severity signature profile which indicates that the subject has, or is at risk of developing, severe COVID-19 can be hospitalized. In another aspect, the subject can be more effectively triaged, as a healthcare facility seeks to give care first to those most in need. For example, the subject can be given a higher priority for treatment by a physician or health care service. In yet another aspect, the subject can be monitored more often, or differently, than a subject who does not receive a severity signature profile which indicates that the subject has, or is likely to develop, severe COVID-19. For example, a score which indicates that the subject has, or is more likely to develop, severe COVID-19 can be contacted via phone calls/text messages/emails or other forms of communication to determine if the subject needs to be hospitalized or receive other forms of treatment or physical monitoring. The subject can be asked to respond to a questionnaire on an hourly, daily, weekly, or any other time frame. The subject can be monitored for temperature, or oxygen levels, or blood markers such as cytokines or other markers of inflammation, either at home or in a hospital or clinical setting. If the subject has not yet been diagnosed with COVID-19, the subject can be tested more often to determine if the subject has acquired COVID-19.

The subject who has undergone the assay to determine the severity signature profile may or may not have already been diagnosed with COVID-19. For example, the subject may have already had a test to determine whether SARS-CoV-2 is present, or whether antibodies to SARS-CoV-2 are present. The subject may or may not have known exposure to COVID-19. The severity signature profile can also be used to determine priority for vaccination. For example, a subject who has a severity signature profile which indicates that the subject is likely to have severe COVID-19 can be prioritized for vaccination. They can also be given more doses, or given the doses more frequently, than a subject whose severity signature profile does not indicate that they are likely to develop severe COVID-19.

A subject who has a severity signature profile which indicates that the subject has, or is likely to develop, severe COVID-19 can be given therapy to treat or prevent COVID-19 or SARS-CoV-2 infection. For example, the subject can be given antiviral therapy, corticosteroids, or monoclonal antibodies. In some cases, the treatments would not have been given to the subject but for the severity signature profile which indicates that such treatment is appropriate. Such treatments can include Remdesivir, Ritonavir-boosted nirmatrelvir, Bebtelovimab, Molnupiravir, Sotrovimab, dexamethasone and other FDA approved therapies.

These treatments can be given in amounts, or for durations, which are different than those given for a subject whose severity signature profile indicates that they are not likely to have, or to develop, severe COVID-19. Given that the high-risk signature is derived primarily from monocytes and neutrophils. Methods to deplete monocytes and neutrophils could be used to change a risk profile from high to low risk thus decreasing the risk of mortality.

In the methods disclosed herein, any one of the following genes can be measured for increased expression (also referred to herein as “upregulated”): PLBD1, TPST1, MCEMP1, IL1R2, HP, FLT3 and S100A12. It is noted that expression levels from 1, 2, 3, 4, 5, 6, or all 7 genes can be measured and used to create a severity signature profile. Furthermore, any combination of these 7 genes can be used to determine the severity signature score.

In some embodiments, PLBD1 is measured for increased expression. In some embodiments, TPST1 is measured for increased expression. In some embodiments, MCEMP1 is measured for increased expression. In some embodiments, IL1R2 is measured for increased expression. In some embodiments, HP is measured for increased expression. In some embodiments, FLT3 is measured for increased expression. In some embodiments, S100A12 is measured for increased expression.

In another aspect, any one of the following genes can be measured for decreased expression (also referred to as “downregulated”): LCK, CAMK2D, NUP43, SLAMF7, LRRC39, ICOS, CD47, LBH, SH2D1A, CNOT6L, METTL8, ETS1, P2RY10, TRAT1, BTN3A1, LARP4, TC2N, GPR183, MORC4, STAT4, LPAR6, CPED1, DOCK10, ARHGAP5, HLA-DPA1, BIRC3, GPR174, CD28, UTRN, CD2, HLA-DPB1, ARL4C, BTN3A3, CXCR6, DYNC2LI1, BTN3A2, ITK, CD96, GBP4, S1PR1, NAP1L2, KLF12, IL7R. It is noted that expression levels from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or all 43 of these genes can be measured and used to create a severity signature profile. Furthermore, any combinations of these 43 genes can be used to determine the severity signature profile.

In some embodiments, LCK is measured for decreased expression. In some embodiments, CAMK2D is measured for decreased expression. In some embodiments, NUP43 is measured for decreased expression. In some embodiments, SLAMF7 is measured for decreased expression. In some embodiments, LRRC39 is measured for decreased expression. In some embodiments, ICOS is measured for decreased expression. In some embodiments, CD47 is measured for decreased expression. In some embodiments, LBH is measured for decreased expression. In some embodiments, SH2D1A is measured for decreased expression. In some embodiments, CNOT6L is measured for decreased expression. In some embodiments, METTL8 is measured for decreased expression. In some embodiments, ETS1 is measured for decreased expression. In some embodiments, P2RY10 is measured for decreased expression. In some embodiments, TRAT1 is measured for decreased expression. In some embodiments, BTN3A1 is measured for decreased expression. In some embodiments, LARP4 is measured for decreased expression. In some embodiments, TC2N is measured for decreased expression. In some embodiments, GPR183 is measured for decreased expression. In some embodiments, MORC4 is measured for decreased expression. In some embodiments, STAT4 is measured for decreased expression. In some embodiments, LPAR6 is measured for decreased expression. In some embodiments, CPED1 is measured for decreased expression. In some embodiments, DOCK10 is measured for decreased expression. In some embodiments, ARHGAP5 is measured for decreased expression. In some embodiments, HLA-DPA1 is measured for decreased expression. In some embodiments, BIRC3 is measured for decreased expression. In some embodiments, GPR174 is measured for decreased expression. In some embodiments, CD28 is measured for decreased expression. In some embodiments, UTRN is measured for decreased expression. In some embodiments, CD2 is measured for decreased expression. In some embodiments, HLA-DPB1 is measured for decreased expression. In some embodiments, ARL4C is measured for decreased expression. In some embodiments, BTN3A3 is measured for decreased expression. In some embodiments, CXCR6 is measured for decreased expression. In some embodiments, DYNC2LI1 is measured for decreased expression. In some embodiments, BTN3A2 is measured for decreased expression. In some embodiments, ITK is measured for decreased expression. In some embodiments, CD96 is measured for decreased expression. In some embodiments, GBP4 is measured for decreased expression. In some embodiments, S1PR1 is measured for decreased expression. In some embodiments, NAP1L2 is measured for decreased expression. In some embodiments, KLF12 is measured for decreased expression. In some embodiments, IL7R is measured for decreased expression.

Also, a score can be obtained by determining the gene expression level of any one or more of the 7 genes that show increased expression, and a score can be obtained for the gene expression level any one or more of the 43 genes that show decreased expression. For example, expression levels from 1, 2, 3, 4, 5, 6, or all 7 genes of the first set can be measured to obtain a first score, and expression levels from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or all 43 of these second set of genes can be measured to obtain second score. These scores can then be combined to obtain a severity signature profile.

The present disclosure makes use of the Scoring Algorithm of Molecular Subphenotypes (SAMS) system. The algorithm was developed to identify novel molecular subphenotypes based on the expression of a predefined set of increased and decreased genes in a given sample. The up and down scores of SAMS are calculated using the product of two variables: the proportion of genes expected to be increased or decreased per patient and their normalized expression levels.

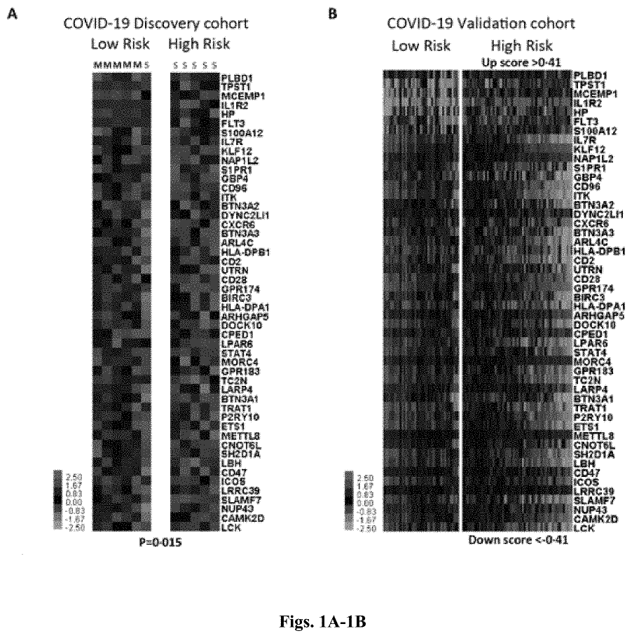

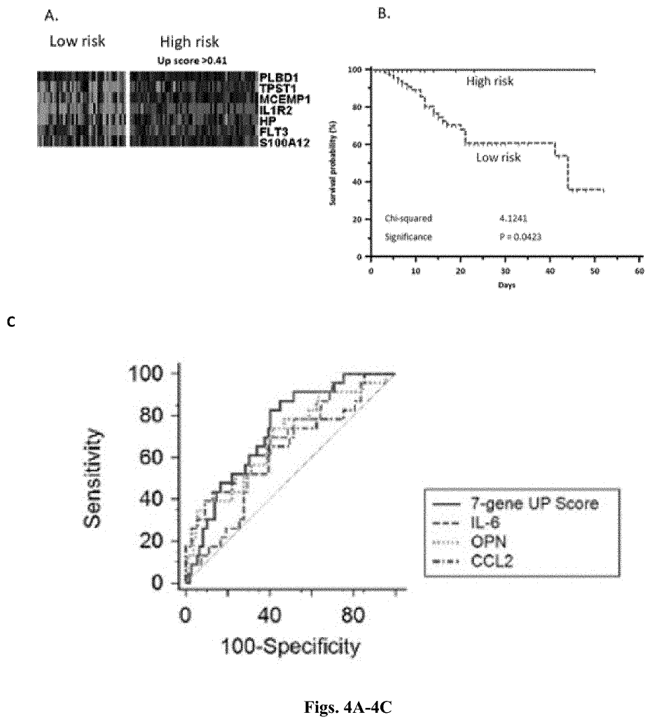

For the method disclosed herein, up and down scores are calculated based on at least one of the fifty genes which increase (or decrease) in expression in those who have, or are likely to have, severe COVID-19. The 7 genes in FIG. 4, which are the same 7 genes that are listed first in FIG. 1, are referred to herein as “up genes,” or genes that increase in expression in those that have, or are likely to have, severe COVID-19. The 43 remaining genes in FIG. 1 (after the first 7 “up genes”) are referred to herein as “down genes,” or genes that decrease in expression in those who have, or who are likely to have, severe COVID-19. The calculation is performed in four steps:

1) Gene normalization: The expression of each gene is normalized (subtracted) to the geometric mean of all the samples in each independent cohort. The geometric mean is specific to each gene and is calculated by taking the nth root of the product of n numbers. This step is performed in order to determine whether the expression of a gene is either increased or decreased in a patient when compared to other patients in the same cohort.

2) Calculation of the proportion of up and down-regulated genes: This can be done using at least 1, but up to all 50, genes disclosed herein. Given that the 50-gene signature is based on seven increased and 43 decreased genes, the proportion of genes expected to be either increased or decreased can be estimated per patient to calculate up and down scores. That is, if patient X has five increased genes out of the seven genes expected to be increased then the proportion of increased genes for this patient is 0.714 (5/7). If the same patient has five decreased genes out of the 43 genes expected to be decreased, then the proportion of decreased genes for the same patient is 0.116 (5/43).

3) Sum of the normalized expression values of increased and decreased genes: The sum of the normalized expression values (calculated in Step 1), is calculated per patient for the entire set of increased genes and for the entire set of decreased genes separately. For patient X in the example above, if the normalized expression values of the five increased genes are 0.213+0.273+0.295+0.485+0.923, then the sum of these expression values is 2.190. If the normalized expression values of the five decreased genes for the same patient are −0.202 (+) −0.140 (+)−0.086 (+)−0.082 (+)−0.066, then the sum of these expression values is −0.578.

4) Calculation of the product between the sum of normalized expression values and the proportion of increased or decreased genes: For this step, the sum of increased genes calculated in Step 3 is multiplied by the proportion of increased genes calculated in Step 2. For patient X in the example above the product between these two variables is 0.714*2.190=1.564; this value is the up score. The same process is followed for the down score calculation and the product between these two variables for patient X is 0.116*−0.578=−0.067; this value is the down score. If a patient does not have any of the seven genes expected to be increased, then the up score is 0. The same is true for patients without any of the 43 genes expected to be decreased.

To determine 50-gene risk profiles in the COVID-19-Discovery cohort, Up scores above the median and Down scores below the median value within this cohort were classified as high-risk. Subjects without this pattern of expression were classified as low risk. In the 50-gene, high-risk group of the COVID-19-Discovery cohort, the lowest Up score (0.41) and the highest Down score (−0.41) were used as cutoffs to identify a 50-gene, high-risk profile (subjects or cells with Up score >0.41 and Down score <−0.41) in the COVID-19-validation, IPF-University of Chicago, IPF-Imperial College London and COVID-19-Single-cell cohort.

Two-sided Fisher's exact test was used to identify differences in disease severity between risk groups in the COVID-19-Discovery cohort. Categorical variables and continuous clinical variables were analyzed using Two-sided Fisher's exact and two-sample t-test, respectively.

As described above, a determination of “high risk” may help guide a physician's treatment of the individual. Alternatively, a determination of “low risk” may also help guide a physician's treatment of the individual.

Time course analysis is also within the scope of the present disclosure. The up scores and down scores of an individual can be determined at two different times. The change in up score and down score can then be used to determine whether the individual fits the high-risk profile. Patients are classified as high risk if their up scores increased by at least 10% (for example 15%, 20%, 30%, 40%, 50, 60%, 70%, 80%, 90%, 100%, and so on) at a later time point while their down scores decreased by at least 10% (for example 15%, 20%, 30%, 40%, 50, 60%, 70%, 80%, 90%, 100%, and so on). In contrast, patients that did not have up scores increase by 10% or greater (for example 15%, 20%, 30%, 40%, 50, 60%, 70%, 80%, 90%, 100%, and so on) and who did not have down scores decrease by 10% or greater (for example 15%, 20%, 30%, 40%, 50, 60%, 70%, 80%, 90%, 100%, and so on), are classified as low risk. Samples may be taken within one month of each other or years apart; for example 15 days, 20 days, 30 days, 1 month, 2 months, 3 months, 4 months, 5 months, 6 months, 8 months, 10 months, 12 months, 14 months, 16 months, 18 months, 20 months, 22 months, 2 years, 2.5 years, 3 years, 4 years, 5 years, 6 years, 7 years, 8 years, 9 years, 10 years, or even longer. By way of non-limiting illustration, examples of certain embodiments of the present disclosure are given below.

Also disclosed herein is a method of treating a subject with severe COVID-19 or at risk of developing severe COVID-19, the method comprising: obtaining a sample from the subject; measuring expression level of at least one of the following genes from the subject: PLBD1, TPST1, MCEMP1, IL1R2, HP, FLT3 and S100A12; measuring expression level of at least one of the following cytokines from the subject: IL-6, CCL2, and/or Osteopontin (OPN); determining a score for the measured gene expression level of the at least two genes from the list of 7 genes, the list of 3 cytokines, or a combination of one or more of both; using the score to obtain a severity signature profile; determining that the severity signature profile is greater than a gene-specific standard; treating the subject for severe COVID-19 or to prevent severe COVID-19.

### Kits

Also disclosed herein are kits for detecting expression of one or more of the 50 genes disclosed herein (listed in FIG. 1). Exemplary kits include nucleic acids configured for specific hybridization to one or more of these genes. In some cases a kit comprises a plurality of such nucleic acids immobilized on a substrate, such as a microarray, welled plate, chip, or other material suitable for microfluidic processing.

In some embodiments, the kit includes nucleic acid and/or polypeptide isolation reagents. In some embodiments, the kit includes one or more detection reagents, for example probes and/or primers for amplification of, or hybridization to, one or more of the 50 genes disclosed herein. In some embodiments, the kit includes primers and probes for control genes, such as housekeeping genes. In some embodiments, the primers and probes for control genes are used. In some embodiments, the probes or primers are labeled with an enzymatic, florescent, or radionuclide label.

In some instances, a kit comprises a nucleic acid polymer (e.g., primer and/or probe) comprising at least about 10 contiguous nucleobases having at least about 70%, 80%, 85%, 90%, 91%, 92%, 93%, 94%, 95%, 96%, 97%, 98% or 99% sequence identity or homology to one or more of the 50 genes disclosed herein.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

In some embodiments, a kit includes one or more additional containers, each with one or more of various materials (such as reagents, optionally in concentrated form, and/or devices) desirable from a commercial and user standpoint for use of described herein. Non-limiting examples of such materials include, but not limited to, buffers, primers, enzymes, diluents, filters, carrier, package, container, vial and/or tube labels listing contents and/or instructions for use and package inserts with instructions for use. A set of instructions is optionally included. In a further embodiment, a label is on or associated with the container. In yet a further embodiment, a label is on a container when letters, numbers or other characters forming the label are attached, molded or etched into the container itself; a label is associated with a container when it is present within a receptacle or carrier that also holds the container, e.g., as a package insert. In other embodiments a label is used to indicate that the contents are to be used for a specific therapeutic application. In yet another embodiment, a label also indicates directions for use of the contents, such as in the methods described herein.

### Systems

Disclosed herein, in some embodiments, is a system for determining a severity signature profile. Therefore, disclosed herein is a system for determining a severity signature profile in a subject, comprising: (a) a computer processing device, optionally connected to a computer network; and (b) a software module executed by the computer processing device to analyze a target nucleic acid sequence(s) of a transcriptomic profile in a sample from a subject. In some instances, the system comprises a central processing unit (CPU), memory (e.g., random access memory, flash memory), electronic storage unit, computer program, communication interface to communicate with one or more other systems, and any combination thereof. In some instances, the system is coupled to a computer network, for example, the Internet, intranet, and/or extranet that is in communication with the Internet, a telecommunication, or data network. In some embodiments, the system comprises a storage unit to store data and information regarding any aspect of the methods described in this disclosure. Various aspects of the system are a product or article or manufacture.

One feature of a computer program includes a sequence of instructions, executable in the digital processing device's CPU, written to perform a specified task. In some embodiments, computer readable instructions are implemented as program modules, such as functions, features, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions are combined or distributed as desired in various environments. In some instances, a computer program comprises one sequence of instructions or a plurality of sequences of instructions. A computer program may be provided from one location. A computer program may be provided from a plurality of locations. In some embodiment, a computer program includes one or more software modules. In some embodiments, a computer program includes, in part or in whole, one or more web applications, one or more mobile applications, one or more standalone applications, one or more web browser plug-ins, extensions, add-ins, or add-ons, or combinations thereof.

The medium, method, and system disclosed herein can comprise one or more databases, or use of the same. In view of the disclosure provided herein, those of skill in the art will recognize that many databases are suitable for storage and retrieval of relative information. Suitable databases include, by way of non-limiting examples, relational databases, non-relational databases, feature oriented databases, feature databases, entity-relationship model databases, associative databases, and XML databases. In some embodiments, a database is internet-based. In some embodiments, a database is web-based. In some embodiments, a database is cloud computing-based. A database may be based on one or more local computer storage devices.

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as genetic or processed cellular material, performed in one facility in a particular location and corresponding data transmitted to another location, or directly to an individual, such as data related to the diagnosis, prognosis, responsiveness to therapy, or the like, in the same or different location or country.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. 50-Gene Risk Profiles in Peripheral Blood Predict COVID-19 Outcomes

### Methods

Study Design and Subjects

Clinical and gene expression data from 50 genes was extracted and analyzed from a COVID-19-Discovery cohort (N=5 mild and N=6 severe cases, single-cell, peripheral blood RNAseq, GEO Accession: GSE1496895), COVID-19-Validation (N=100, bulk peripheral blood leukocytes RNAseq, GEO Accession: GSE1571036), COVID-19-Single cell (N=7 subjects, N=145 single-cell measurements, Single-cell RNAseq, GEO accession: GSE1507287), IPF-University of Chicago (N=45, Affymetrix Human Exon 1.0 ST Array of bulk peripheral blood mononuclear cells RNA, GEO Accession: GSE282213) and IPF-Imperial College London (N=55, Affymetrix Human Gene 1.1 ST Array of whole blood RNA) cohorts. Transcriptomics data collection from all cohorts has been previously described (Herazo-Maya 2013; Lee 2020; Overmyer 2020; and Herazo 2011). Studies were approved by Institutional Review Boards at each institution.

Data Extraction, Pre-Processing and Statistical Analysis

All analyses were performed in R software (version 4.0.2) (Team 2020). For the COVID-19-Discovery cohort, the R package “Seurat” was used to preprocess the feature-barcode matrices of the single cell RNAseq data. Cells expressing less than 200 genes or more than 15% of mitochondrial genes of their total gene expression were excluded. Genes expressed in less than 10 cells were also excluded from the analysis. NormalizeData function was used to normalize gene expression levels. Subject-level expression profile was estimated using the average expression level across all cells. For the bulk RNA-seq data in the COVID-19 Validation cohort, Transcripts Per Million (TPM) matrix was analyzed using log(1+TPM) to normalize gene expression levels. For the COVID-19-Single cell dataset, preprocessed and normalized data were provided directly according to the published paper (Herazo 2011).

The Scoring Algorithm for Molecular Subphenotypes (SAMS) (Herazo-Maya 2017) was applied as previously described. SAMS, Up and Down scores were calculated in each cohort using the product of two variables: the proportion of genes expected to be increased or decreased per subject (or single cells) and their normalized expression levels (Herazo-Maya 2017). In this study, Up and Down scores were calculated in all cohorts based on the expression levels of seven increased genes (PLBD1, TPST1, MCEMP1, IL1R2, HP, FLT3, S100A12) and 43 decreased genes (LCK, CAMK2D, NUP43, SLAMF7, LRRC39, ICOS, CD47, LBH, SH2D1A, CNOT6L, METTL8, ETS1, C2orf27A, P2RY10, TRAT1, BTN3A1, LARP4, TC2N, GPR183, MORC4, STAT4, LPAR6, CPED1, DOCK10, ARHGAP5, HLA-DPA1, BIRC3, GPR174, CD28, UTRN, CD2, HLA-DPB1, ARL4C, BTN3A3, CXCR6, DYNC2LI1, BTN3A2, ITK, SNHG1, CD96, GBP4, S1PR1, NAP1L2, KLF12, IL7R) from a 52-gene signature previously found to be predictive of IPF mortality. Two non-coding transcripts (SNHG1, C2orf27A) of the original 52-gene signature were excluded because they were not present consistently across datasets.

To determine 50-gene risk profiles in the COVID-19-Discovery cohort, Up scores above the median and Down scores below the median value within this cohort were classified as high-risk. Subjects without this pattern of expression were classified as low risk. In the 50-gene, high-risk group of the COVID-19-Discovery cohort, the lowest Up score (0.41) and the highest Down score (−0.41) were used as cutoffs to identify a 50-gene, high-risk profile (subjects or cells with Up score >0.41 and Down score <−0.41) in the COVID-19-validation, IPF-University of Chicago, IPF-Imperial College London and COVID-19-Single-cell cohort.

Two-sided Fisher's exact test was used to identify differences in disease severity between risk groups in the COVID-19-Discovery cohort. Categorical variables and continuous clinical variables were analyzed using Two-sided Fisher's exact and two-sample t-test, respectively. The Area Under the Curve (AUC) was used to assess the prediction accuracy of the 50-gene risk profiles to determine ICU admission, use of mechanical ventilation and hospital mortality in the COVID-19-Validation cohort.

Kaplan-Meier curves were used to evaluate the association between 50-gene risk profiles and Mortality in IPF cohorts.

### 50-Gene Risk Profiles in Peripheral Blood Predict COVID-19 Outcomes

The COVID-19-Discovery cohort included five and six individuals with mild and severe COVID-19, respectively. To identify 50-gene risk profiles in this cohort, SAMS Up and Down scores were calculated for each individual. All the subjects with a 50-gene, high-risk profile had severe COVID-19 while 83.3% of subjects with a low-risk profile had mild COVID-19 (P=0.015) (FIG. 1A). SAMS cutoffs derived from the COVID-19-Discovery cohort (Up score >0.41 and Down score <−0.41) distinguished High versus Low-risk subjects in the COVID-19-Validation cohort (FIG. 1B). High-risk subjects in the validation cohort were significantly older (64.8 versus 55 years), had higher APACHE-II severity score (22.5 versus 14.1) and Charlson Comorbidity Index (4 versus 2.3). They also had higher C-reactive protein (165.7 mg/l versus 101.3 mg/1), and Ferritin levels (1215.6 ng/ml versus 497 ng/ml) (Table 2). A 50-gene, high-risk profile predicted ICU admission (AUC:0.77, 95% CI:0.686-0.844, P<0.0001), mechanical ventilation (AUC:0.75, 95% CI:0.67-0.827, P<0.0001) and in-hospital mortality (AUC:0.74, 95% CI:0.678-0.815, P<0.0001) in the COVID-19-Validation cohort (Table 1). The addition of Age and Comorbidities, measured by the Charlson index, to 50-gene risk profiles modestly improved the mortality prediction accuracy of the genomic classifier by 3% (AUC went from 0.74 to 0.77). High-risk patients spent more days on mechanical ventilation (21.9 versus 15.5 days) and more days hospitalized (21.1 versus 9 days) compared to low risk patients. Only one patient in the 50-gene, low-risk profile group died while 23 patients in the 50-gene, high-risk profile group died during hospitalization (P=0.000009) (Table 1).

### 50-Gene Risk Profiles in COVID-19 are Predictive of Mortality in IPF

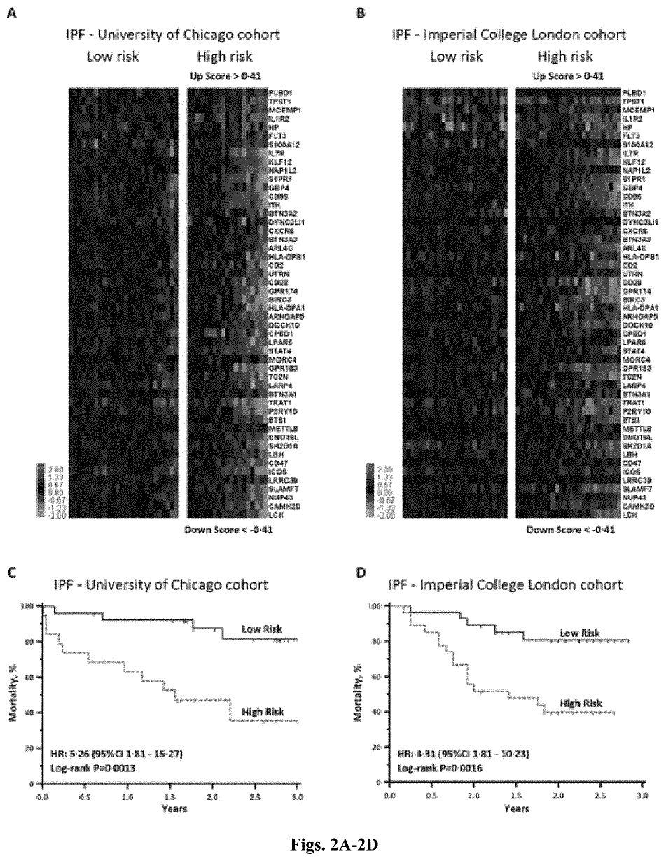

To determine whether same SAMS cutoffs used to distinguish a 50-gene, high-risk profile in COVID-19 could also be applied to predict IPF mortality, reanalyzed peripheral blood 50-gene expression data were extracted and analyzed from two independent IPF cohorts (IPF-University of Chicago Cohort and IPF-Imperial College London). An Up Score >0.41 and a Down Score <−0.41 distinguished 50-gene high versus low-risk profiles in both IPF cohorts (FIGS. 2A and 2B). 50-gene risk profiles were significantly predictive of mortality in the IPF-University of Chicago (HR:5.26, 95% CI:1.81-15.27, P=0.0013) and IPF-Imperial College London (HR:4.31, 95% CI:1.81-10.23, P=0.0016) cohorts (FIGS. 2C and 2D). These results confirmed our previous findings (Herazo 2017) and indicate an overlapping outcome-associated transcriptomic signature between COVID-19 and IPF.

### Single-Cell Analysis in COVID-19 Reveals the Cellular Sources of the 50-Gene Risk Profiles

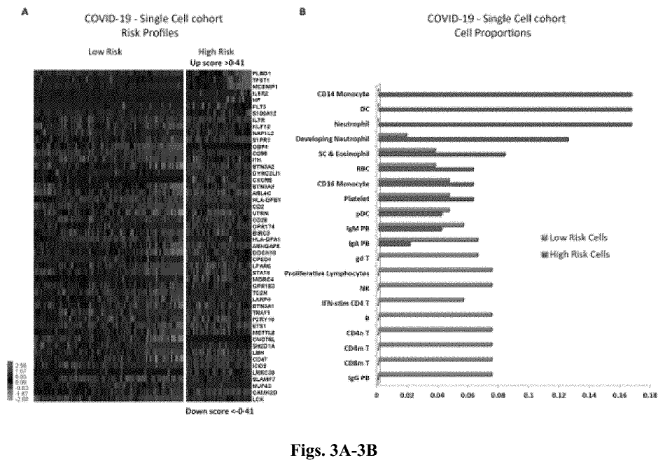

A cellular-source cohort (Seven COVID-19 subjects and 145 single cells) (Scott 2019) was used to identify the cellular origin of the 50-gene risk profiles. SAMS cutoffs derived from the COVID-19-Discovery cohort (Up score>0.41 and a Down<−0.41) classified 47 cells with a high-risk profile and 108 cells with a low-risk profile (FIG. 3A). The estimated proportion of 50-gene expressing cells in the high-risk profile mainly included CD14⁺ monocytes (16.7%), dendritic cells (16.7%), neutrophils (16.7%), developing neutrophils (12.5%), eosinophils (8.3%), myeloid cells (6.25%), CD16⁺ monocytes (6.25%) and platelets (6.25%) while 50-gene expressing cells in the low-risk profile cells mainly included IgG producing Plasmablasts (7.48%) mature (7.48%) and naïve (7.48%) CD4 T cells, CD8 T cells (7.48%), B cells (7.48%), NK cells (7.48%), proliferative lymphocytes (7.48%) and gamma/delta T cells (6.54%) (FIG. 3B). The full list of proportion of 50-gene expressing cells can be seen in the online supplement (Table 3). These findings provide evidence of the cellular source of 50-gene expression changes in peripheral blood and point at specific cell-types potentially associated with increased risk of mortality and other poor outcomes in COVID-19.

TABLE 1
Prediction accuracy of 50-gene risk profiles to predict outcomes in COVID-19

| | ICU admission | Mechanical Ventilation | In-Hospital Mortality |
|---|---|---|---|
| Prediction models | (AUC, 95% CI) | (AUC, 95% CI) | (AUC, 95%) |
| 50-Gene Risk Profiles | 0.77, | 0.75, | 0.74, |
| (High versus Low) | 95% CI (0.686-0.844) | 95% CI (0.67-0.827) | 95% CI (0.678-0.815) |
| Charlson Index | 0.54, | 0.48, | 0.69, |
| | 95% CI (0.432-0.648) | 95% CI (0.37-0.576) | 95% CI (0.553-0.797) |
| 50-Gene Risk Profiles and | 0.78, | 0.79, | 0.77, |
| Charlson index | 95% CI (0.597-0.847) | 95% CI (0.634-0.864) | 95% CI (0.531-0.866) |

TABLE 2
Clinical Variables of the COVID-19-Validation cohort

| | High risk | | Low Risk | | Sample | |
|---|---|---|---|---|---|---|
| Clinical Variables | N = 59 | | N = 41 | | Size | P value |
| Age (Mean ± SD) | 64.8 | (14.5) | 55 | (16.6) | 99 | <0.01* |
| Gender | | | | | 100 | |
| Males (%) | 36 | (61%) | 26 | (63.4%) | | 0.83 |
| Females (%) | 23 | (39%) | 15 | (36.6%) | | |
| ICU admission | | | | | 00 | |
| Yes (%) | 43 | (72.9%) | | (17.1%) | | <0.001* |
| No (%) | 16 | (27.1%) | 34 | (82.9%) | | |
| Mechanical Ventilation | | | | | 100 | <0.001* |
| Yes (%) | 37 | (62.7%) | 5 | (12.2%) | | |
| No (%) | 22 | (37.3%) | 36 | (87.8%) | | |
| In-Hospital Mortality | | | | | 100 | <0.001* |
| Yes (%) | 23 | (39%) | 1 | (2.4%) | | |
| No (%) | 36 | (61%) | 40 | (97.6%) | | |
| Ventilator-Free Days - | 15.5 | (12.3) | 21.9 | (6.6) | 100 | <0.001* |
| 28 days follow up | | | | | | |
| (Mean ± SD) | | | | | | |
| Hospital length of stay | 21.1 | (12.7) | 9 | (8.9) | 100 | <0.001* |
| (Mean ± SD) | | | | | | |
| APACHE II | 22.5 | (8.1) | 14.1 | (3.5) | 57 | <0.01* |
| (Mean ± SD) | | | | | | |
| Charlson Comorbidity Index | 4 | (2.6) | 2.3 | (1.9) | 100 | <0.001* |
| (Mean ± SD) | | | | | | |
| Ferritin (ng/ml) (Mean ± SD) | 1215.6 | (1294.6) | 497 | (403.8) | 94 | <0.01* |
| C-reactive protein (mg/l) | 165.7 | (107.9) | 101.3 | (83.6) | 92 | <0.01* |
| (Mean ± SD) | | | | | | |

TABLE 3
Estimated proportions of 50-gene expressing
cells with High versus Low-risk profiles

| Cell Type | Low Risk (%) | High Risk (%) |
|---|---|---|
| IgG PB | 7.48 | 0 |
| CD8m T | 7.48 | 0 |
| CD4m T | 7.48 | 0 |
| CD4n T | 7.48 | 0 |
| B | 7.48 | 0 |
| NK | 7.48 | 0 |
| Proliferative | 7.48 | 0 |
| Lymphocytes | | |
| gd T | 6.54 | 0 |
| IFN-stim CD4 T | 5.61 | 0 |
| IgA PB | 6.54 | 2.08 |
| IgM PB | 5.61 | 4.17 |
| pDC | 4.67 | 4.17 |
| Platelet | 4.67 | 6.25 |
| CD16 Monocyte | 4.67 | 6.25 |
| Myeloid cells | 3.74 | 6.25 |
| SC & Eosinophil | 3.74 | 8.33 |
| Developing | 1.87 | 12.5 |
| Neutrophil | | |
| Neutrophil | 0 | 16.67 |
| DC | 0 | 16.67 |
| CD14 Monocyte | 0 | 16.67 |

B: B Cell, CD4m T: Memory CD4 T Cell, CD4n T: Naive CD4 T Cell, CD8m T: Memory CD8 T Cell, DC: Conventional Dendritic Cell, gd T: Gamma Delta T cells, IFN-stim CD4 T: Interferon-stimulated CD4 T cell, IgA PB: IgA (Immunoglobulin-A) Plasmablast, IgG PB: IgG (Immunoglobulin-G) Plasmablast, IgM PB: IgM (Immunoglobulin-M) Plasmablast, NK: Natural Killer Cell, pDC: Plasmacytoid Dendritic Cell, Myeloid cells, SC & Eosinophil: Stem Cells and Eosinophil.

### Discussion

In this study, it is shown that a high-risk, 50-gene profile, previously shown to predict IPF mortality is also predictive of worse outcomes in COVID-19 patients. The transcriptomic overlap captured in different cohorts and experimental settings suggests a remarkably conserved systemic gene expression signature evoked by COVID-19 and IPF. Moreover, this overlapping profile combined with the observed pathological and radiological surrogates of pulmonary fibrosis shown by some severe COVID-19 patients suggests that both diseases share, to some extent, common host response features. The single-cell RNA sequencing data in COVID-19 subjects points at the cells expressing the 50 genes predictive of poor disease outcomes. These data suggest that CD14⁺ monocytes and neutrophils, among other cells, are critical regulators of the high-risk profile. In SARS-CoV-2 infected primates, increased circulating levels of classical and non-classical monocytes, and neutrophilic migration to the lungs has been associated with poor disease outcomes. In humans, reports have shown that severe COVID-19 is associated with elevated numbers of neutrophil precursors and circulating levels of CD14⁺ monocytes with high expression of alarmins S100A8/9/12 and low expression of HLA-DR (Schulte-Schrepping 2020). The present analysis is consistent with that data, and a recent report indicates that serum calprotectin, which belongs to the S100 protein family, is associated with the diagnosis of IPF and correlates with diffusing capacity for carbon monoxide (DLCO) and with the composite physiologic index (CPI) (Machahua 2021). Moreover, previous evidence indicates that S100A9 is elevated in bronchoalveolar lavage fluid from IPF patients in comparison with healthy controls (Nara 2012) and increased circulating levels of CD14⁺ monocytes were found to be predictive of mortality in IPF and other fibrotic lung diseases (Scott 2019).

The single-cell RNA sequencing data shows increased proportion of CD4 and CD8 T lymphocytes and immunoglobulin-producing plasmablasts in individuals with low-risk transcriptomic profile, suggesting an association between a strong T cell response (Chen 2020: Atyeo 2020) and better disease outcomes (Zhang 2020). This finding is consistent with recent data indicating that severe COVID-19 infection induces a distinct inflammatory program characterized by suppression of the innate immune system in the periphery and that milder cases evoke a more robust T cell response (Arunachalam 2020).

The biomarker implications of this discovery are significant since the identification of 50-gene risk profiles in COVID-19, in addition to clinical variables, can aid with healthcare utilization such as triage of patients to the most appropriate location (home, ward, ICU), reduce hospital length-of-stay, allow for proper allocation of limited resources including mechanical ventilators and reduce the cost of inappropriate hospitalization. It can also facilitate the early identification of patients likely to deteriorate and resolve specific transcriptomic sub-phenotypes that are amenable to certain treatments. For example, while corticosteroids are currently recommended for hospitalized COVID-19 patients (Group 2020) these medications have demonstrated different effects in sepsis patients depending on circulating bulk transcriptomic profiles (Antcliffe 2019), thus, the 50-gene, high-risk profile can facilitate the identification of patients that are more likely to respond to COVID-19 targeted therapies such as corticosteroids and others (Shrestha 2020). Given that COVID-19 outcome is predominantly driven by the host response to the infection (Zhang 2020) which appears to be shared by IPF patients to some extent, this signature supports the rationale to investigate the use of IPF-targeted antifibrotic medications (King 2014; Richeldi 2014) to prevent short- and long-term sequela of COVID-19.

In conclusion, peripheral blood, 50-gene risk profiles predicts ICU admission, need for mechanical ventilation and in-hospital mortality in COVID-19 and overlaps a signature known to predict poor IPF outcomes. The cellular sources of these gene expression changes suggest common mechanisms implicating adaptive immune response in both diseases. A 50-gene, risk profile test in peripheral blood is a potentially useful biomarker to predict COVID-19 mortality and morbidity.

The compositions, devices, systems, and methods of the appended claims are not limited in scope by the specific compositions, devices, systems, and methods described herein, which are intended as illustrations of a few aspects of the claims. Any compositions, devices, systems, and methods that are functionally equivalent are intended to fall within the scope of the claims. Various modifications of the compositions, devices, systems, and methods in addition to those shown and described herein are intended to fall within the scope of the appended claims. Further, while only certain representative compositions, devices, systems, and method steps disclosed herein are specifically described, other combinations of the compositions, devices, systems, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

Unless defined otherwise, all technical and scientific terms used herein have the same meanings as commonly understood by one of skill in the art to which the disclosed invention belongs. Publications cited herein and the materials for which they are cited are specifically incorporated by reference.

### REFERENCES

1. Carsana L, Sonzogni A, Nasr A, et al. Pulmonary post-mortem findings in a series of COVID-19 cases from northern Italy: a two-centre descriptive study. Lancet Infect Dis 2020; 20(10): 1135-40.
2. Ackermann M, Verleden S E, Kuehnel M, et al. Pulmonary Vascular Endothelialitis, Thrombosis, and Angiogenesis in Covid-19. The New England journal of medicine 2020; 383(2): 120-8.
3. Grillo F, Barisione E, Ball L, Mastracci L, Fiocca R. Lung fibrosis: an undervalued finding in COVID-19 pathological series. Lancet Infect Dis 2020.
4. Tian S, Xiong Y, Liu H, et al. Pathological study of the 2019 novel coronavirus disease (COVID-19) through postmortem core biopsies. Mod Pathol 2020; 33(6): 1007-14.
5. Zhou S, Wang Y, Zhu T, Xia L. CT Features of Coronavirus Disease 2019 (COVID-19) Pneumonia in 62 Patients in Wuhan, China. AJR American journal of roentgenology 2020; 214(6): 1287-94.
6. Spagnolo P, Balestro E, Aliberti S, et al. Pulmonary fibrosis secondary to COVID-19: a call to arms? Lancet Respir Med 2020; 8(8): 750-2.
7. Herazo-Maya J D, Noth I, Duncan S R, et al. Peripheral blood mononuclear cell gene expression profiles predict poor outcome in idiopathic pulmonary fibrosis. Science translational medicine 2013; 5(205): 205ra136.
8. Herazo-Maya J D, Sun J, Molyneaux P L, et al. Validation of a 52-gene risk profile for outcome prediction in patients with idiopathic pulmonary fibrosis: an international, multicentre, cohort study. Lancet Respir Med 2017; 5(11): 857-68.
9. Lee J S, Park S, Jeong H W, et al. Immunophenotyping of COVID-19 and influenza highlights the role of type I interferons in development of severe COVID-19. Sci Immunol 2020; 5(49).
10. Overmyer K A, Shishkova E, Miller I J, et al. Large-Scale Multi-omic Analysis of COVID-19 Severity. Cell Syst 2020.
11. Wilk A J, Rustagi A, Zhao N Q, et al. A single-cell atlas of the peripheral immune response in patients with severe COVID-19. Nature medicine 2020; 26(7): 1070-6.
12. Molyneaux P L, Willis-Owen S A G, Cox M J, et al. Host-Microbial Interactions in Idiopathic Pulmonary Fibrosis. American journal of respiratory and critical care medicine 2017; 195(12): 1640-50.
13. Team R C. R: A language and environment for statistical computing. R Foundation for Statistical Computing, Vienna, Austria. 2020.
14. Fahlberg M D, Blair R V, Doyle-Meyers L A, et al. Cellular events of acute, resolving or progressive COVID-19 in SARS-CoV-2 infected non-human primates. Nat Commun 2020; 11(1): 6078.
15. Schulte-Schrepping J, Reusch N, Paclik D, et al. Severe COVID-19 Is Marked by a Dysregulated Myeloid Cell Compartment. Cell 2020; 182(6): 1419-40 e23.
16. Machahua C, Guler S A, Horn M P, et al. Serum calprotectin as new biomarker for disease severity in idiopathic pulmonary fibrosis: a cross-sectional study in two independent cohorts. BMJ Open Respir Res 2021; 8(1).
17. Hara A, Sakamoto N, Ishimatsu Y, et al. S100A9 in BALF is a candidate biomarker of idiopathic pulmonary fibrosis. Respiratory medicine 2012; 106(4): 571-80.
18. Scott M K D, Quinn K, Li Q, et al. Increased monocyte count as a cellular biomarker for poor outcomes in fibrotic diseases: a retrospective, multicentre cohort study. Lancet Respir Med 2019; 7(6): 497-508.
19. Chen Z, John Wherry E. T cell responses in patients with COVID-19. Nat Rev Immunol 2020; 20(9): 529-36.
20. Atyeo C, Fischinger S, Zohar T, et al. Distinct Early Serological Signatures Track with SARS-CoV-2 Survival. Immunity 2020; 53(3): 524-32 e4.
21. Zhang X, Tan Y, Ling Y, et al. Viral and host factors related to the clinical outcome of COVID-19. Nature 2020; 583(7816): 437-40.
22. Arunachalam P S, Wimmers F, Mok C K P, et al. Systems biological assessment of immunity to mild versus severe COVID-19 infection in humans. Science 2020; 369(6508): 1210-20.
23. Group R C, Horby P, Lim W S, et al. Dexamethasone in Hospitalized Patients with Covid-19—Preliminary Report. The New England journal of medicine 2020.
24. Antcliffe D B, Burnham K L, Al-Beidh F, et al. Transcriptomic Signatures in Sepsis and a Differential Response to Steroids. From the VANISH Randomized Trial. American journal of respiratory and critical care medicine 2019; 199(8): 980-6.
25. Shrestha G S, Paneru H R, Vincent J L. Precision medicine for COVID-19: a call for better clinical trials. Critical care 2020; 24(1): 282.
26. King T E, Jr., Bradford W Z, Castro-Bernardini S, et al. A phase 3 trial of pirfenidone in patients with idiopathic pulmonary fibrosis. The New England journal of medicine 2014; 370(22): 2083-92.
27. Richeldi L, du Bois R M, Raghu G, et al. Efficacy and safety of nintedanib in idiopathic pulmonary fibrosis. The New England journal of medicine 2014; 370(22): 2071-82.

### Example 2. Further Analysis of Gene Risk Profiles for Covid-19

The COVID-19 pandemic has so far caused more than 4.5 million deaths worldwide. Mortality is primarily driven by the development of Acute Respiratory Distress Syndrome (ARDS). Autopsy data from patients dying early after ARDS development demonstrate diffuse alveolar damage, endothelial injury, thrombosis, and angiogenesis. Longer disease courses associate with features of fibrotic lung disease including tissue remodeling, fibroblast proliferation, airspace obliteration, micro-honeycombing and extracellular matrix deposition. Severe disease is also associated with long-term dyspnea, chronic oxygen use, impaired pulmonary function, and fibrotic changes on computed tomography scans of the chest. At the single-cell gene expression level in lung tissue, COVID-19 patients with severe disease have patterns similar to end-stage pulmonary fibrosis without persistent SARS-CoV-2 infection suggesting that some individuals develop accelerated lung fibrosis after resolution of the active infection, a phenomenon that is associated with significant morbidity and mortality. The clinical course of COVID-19 is highly variable and unpredictable. Mortality, treatment response and long-term sequelae are difficult to predict in COVID-19.

A 50-Gene risk profile was identified in peripheral blood predictive of ICU admission, mechanical ventilation, and in-hospital mortality in COVID-19 patients. The mortality prediction accuracy of these gene profiles was previously shown in patients with Idiopathic Pulmonary Fibrosis (IPF) suggesting shared profibrotic mechanisms between COVID-19 and IPF. Data suggests that host resilience and improved survival in COVID-19 is associated with the overexpression of 43 genes in CD4⁺, CD8⁺ T lymphocytes, IgG producing plasmablasts, B cells, NK and gamma delta T cells while mortality and poor outcomes is associated with the overexpression of seven genes in monocytes, dendritic cells, and neutrophils.

Based on this data, the 50-Gene risk profiles and proteins derived from circulating immune cell subsets can predict how COVID-19 patients progress in response to the infection and treatments, particularly in terms of acute and chronic respiratory impairment. In addition, identifying the treatments that moderate these gene expression changes and improve clinical outcomes can lead to a precision medicine-based approach to monitor and individualize therapy.

### Introduction

COVID-19, the disease caused by SARS-CoV-2 infection, emerged in December 2019 in Wuhan, China and has caused more than 4.5 million deaths worldwide according to the World Health Organization. In 2021, the state of Florida is one of the leading states in COVID-19 infections and hospitalizations. The clinical course of COVID-19 is highly variable and unpredictable. According to The Chinese Center for Disease Control and Prevention the majority of infected patients (80%) experience mild disease (with either no or only mild pneumonia), 14% develop severe disease (with dyspnea, hypoxia or greater than 50% lung involvement on imaging tests) and 5% develop critical disease (characterized by respiratory failure, systemic shock or multi-organ failure). Approximately 2-3% of the patients with severe disease progress and die. Current treatment options for COVID-19 include the antiviral Remdesivir and Dexamethasone. The FDA has issued Emergency Use Authorization (EUA) for several monoclonal antibody therapies such as Casirivimab and Imdevimab and, Sotrovimab for the treatment of mild or moderate COVID-19. FDA has also issued EUA for immune modulators such as Baricitinib and Tocilizumab. While these therapies may be effective to treat COVID-19, it is unclear which patients may have a better response to each one of these treatments. Molecular profiling is a tool that can be used to facilitate drug selection, monitoring duration of therapy, and identifying the patients that may respond to certain treatments. In addition, molecular profiling can also facilitate the triage of patients to the most appropriate location such as home, ward or intensive care unit, allocate limited resources, reduce hospital length-of-stay and eliminate the cost of inappropriate hospitalization. Most importantly, it can help identify patients at risk of dying before the disease progresses which can have a positive impact in mortality and ultimately save countless of lives. In the example, a 50-Gene risk profile was investigated in peripheral blood that can be used as a molecular profiling tool in COVID-19.

### 50-Gene Risk Profiles in Peripheral Blood can Predict COVID-19 Mortality and Reflects Single-Cell Gene Expression Changes.

While autopsy data from COVID-19 patients dying early on after ARDS development demonstrates diffuse alveolar damage, endothelial injury, thrombosis, and angiogenesis; longer disease courses associate with long-term sequelae and features of Interstitial Lung Disease (ILD). These features include tissue remodeling, fibroblast proliferation, airspace obliteration, micro-honeycombing and extracellular matrix deposition. Moreover, radiological surrogates of lung fibrosis, including sub-pleural reticulation and fibrotic streaks have also been described in COVID-19. While an association between COVID-19-induced ARDS and risk for ILD development has been suggested, no research has focused on evaluating shared immune gene expression profiles between COVID-19 and Idiopathic Pulmonary Fibrosis (IPF) patients and how these molecular profiles could be used to advance the field of precision medicine in COVID-19. Previous work has identified and validated a peripheral blood gene expression signature predictive of mortality in IPF. This signature was predictive of disease severity and poor outcomes in COVID-19 patients from two independent cohorts. Results with COVID-19 showed that peripheral blood, 50-Gene risk profiles (High vs. Low) discriminated severe from mild COVID-19 in the discovery cohort and significantly predicted ICU admission, need for mechanical ventilation, and in-hospital mortality in the COVID-19 validation cohort. Single-cell RNA sequencing analysis (scRNA-seq) demonstrated that 50-Gene expressing cells with a high-risk profile mainly included monocytes, dendritic cells (DCs), and neutrophils, while low-risk profile-expressing cells included CD4⁺, CD8⁺ T lymphocytes, IgG producing plasmablasts, B cells, NK, and gamma/delta T cells. Taken together, these results show that 50-Gene risk profiles represent aberrant immune responses associated with lung injury, progression, and repair in COVID-19.

### Aberrant Immune Responses and COVID-19 Progression

Several studies have focused on the identification of aberrant immune responses associated with COVID-19 progression. For example, increased circulating levels of classical and non-classical monocytes, and neutrophilic migration to the lungs has been associated with poor disease outcomes in SARS-CoV-2 infected primates. In humans, reports have shown that severe COVID-19 is associated with elevated numbers of neutrophil precursors and circulating levels of CD14⁺ monocytes with high expression of alarmins S100A8/9/12 and low expression of HLA-DR. A recent study combined single-cell transcriptome and surface proteome data from PBMC from individuals with asymptomatic, mild, moderate, severe and critical COVID-19 across three UK centers. The authors found that proliferating monocytes and DCs expressing MKI67 and TOP2A were increased with disease severity. CD14⁺ monocytes were the only population to change significantly with symptom duration. In a different study, evaluating lung tissue obtained from cryobiopsies from patients with moderate COVID-19 and parenchymal lung involvement, irregular clusters of mononuclear cells were demonstrated within alveolar spaces. These cells, characterized as macrophages by the CD68⁺, CD11c⁺, and CD14⁺ immunophenotype, were smaller than typical alveolar macrophages and exhibited an unusual “hybrid” phenotype including dendritic-cell markers (DC-Lamp/CD208, CD206, and CD123/IL3AR). These results confirm the relevance of results showing that monocytes, dendritic cells, and neutrophils are not only potential drivers of COVID-19 progression, but they seem to abnormally express genes of the 50-Gene signature. scRNA-seq data analysis also showed increased proportion of CD4 and CD8 T lymphocytes and immunoglobulin-producing plasmablasts in individuals with a low-risk genomic profile, suggesting an association between a strong T cell response and better disease outcomes.

Recent studies have found that severe COVID-19 infections are associated with gene variants on chromosome 3 (3p21.31) and chromosome 9 (9q34.2)(25), ApoE e4 genotype, and loss-of-function variants on X-chromosomal TLR7. While these findings are relevant to understand COVID-19 severity, the clinical significance of these genetic variants is still debatable and limited by the fact that they cannot be used to monitor disease progression or predict treatment response. In addition to the identification of genetic variants, several authors have demonstrated that patients with moderate or severe COVID-19 have increased levels of inflammatory and profibrotic cytokines in peripheral blood compared to patients with mild COVID-19 or controls. While the inflammatory interleukins IL-1β, IL-6, IL-8, IL-10, IL-13, TNF-α, EN-RAGE, and profibrotic cytokines CCL2, CXCL10, Osteopontin, MMP1, VEGF-A and TGF-β have been shown to be either associated with disease severity, progression, or mortality in COVID-19, investigators have not studied whether these protein biomarkers can be used in combination with genetic variants or genomic profiling to improve outcome prediction or treatment response in COVID-19. Here, a 50-Gene risk profile was identified that are predictive of COVID-19 mortality.

While immune aberrations have been extensively studied during acute COVID-19, they have not been well characterized during post COVID-19, especially in patients with evidence of residual lung fibrosis. The presence of long-term sequela post COVID-19 has been well described ranging from dyspnea, the most common post COVID-19 symptom, chronic oxygen use and fibrotic lung disease. The Swiss COVID-19 lung study, a multicenter prospective cohort investigating pulmonary sequelae of COVID-19 demonstrated that severe/critical disease was associated with impaired pulmonary function, i.e. diffusing capacity of the lung for carbon monoxide (D_{LCO}) % predicted, reduced 6-min walk distance (6MWD) and exercise-induced oxygen desaturation when compared to mild/moderate disease. A reduction in diffusion capacity is the most reported physiologic impairment in post COVID-19, with values directly related to the severity of acute illness. Fibrotic changes on computed tomography scans of the chest, consisting primarily of reticulations or traction bronchiectasis, were observed 3 months after hospital discharge in approximately 25 and 65% of survivors in cohort studies of mild-to-moderate cases and in most of severe cases. Others have also shown lung fibrotic changes on high resolution CT scan (HRCT) images in mechanically ventilated survivors of COVID-19. In a compelling analysis of lung tissue from five cases with severe COVID-19-associated pneumonia, including two autopsy specimens and three specimens from explanted lungs of recipients of lung transplantation, showed histopathologic and single-cell RNA expression patterns similar to end-stage pulmonary fibrosis without persistent SARS-CoV-2 infection, suggesting that some individuals develop accelerated lung fibrosis after resolution of the active infection. While there is significant research demonstrating the presence of long-term sequalae post COVID-19, very few studies have addressed the cellular and molecular mechanisms associated with the exaggerated fibrotic response seen in some COVID-19 survivors.

Currently, most hospitalized patients with COVID-19 get a similar drug regimen that includes dexamethasone, remdesivir and occasionally, antibiotics. Unfortunately, there is not a reliable way to distinguish the patients who will respond to these treatments from those who will progress and die from the disease.

The current results challenge the current evaluation and treatment paradigm in COVID-19 by demonstrating that 50-Gene risk profiles can predict outcomes in patients before the disease progresses and before it is too late for patients to have a meaningful recovery.

The outcome prediction accuracy of 50-Gene risk profiles in COVID-19 is investigated as is the association of these genes with treatment response by applying multiplexed, color-coded probe pairs (nCounter analysis system, Nanostring). The nCounter system captures and counts individual mRNA transcripts by the hybridization of mRNA with a sequence-specific probe containing a biotin tag, and a sequence complementary to the target mRNA that contains a coupled color-coded tag which provides the detection signals. This system has the same sensitivity of qRT-PCR and can validate up to 800 genes in a single tube using very low amounts of RNA material. The nCounter system has several advantages over RNA-seq including less cost per sample, high-throughput performance (up to 36 samples can be processed per day) and provides transcript counts the same day of sample processing, minimizing data analysis time. To identify 50-Gene risk profiles in COVID-19 patients, the Scoring Algorithm of Molecular Subphenotypes (SAMS) is used, a powerful and highly reproducible classification algorithm of gene expression data which has been recently validated in COVID-19. To determine the cellular source of 50-Gene risk profiles in acute COVID-19 and post COVID-19 pneumonia, Chromium 10× is used to apply a novel approach that combines single-cell transcriptomics and cell surface proteomics.

### Results

### 50-Gene Risk Profiles Predict COVID-19 Severity and Poor Outcomes in a Discovery and Validation Cohort

Gene expression and clinical data were analyzed from a COVID-19 Discovery cohort (N=8 subjects, Single-cell, peripheral blood RNA sequencing data, GEO Accession: GSE149689) in a retrospective, multicenter cohort study. PBMC were obtained twice from three of these subjects at two different time points during hospitalization. PBMC specimens from patients with COVID-19 were assigned to severe (N=6) or mild (N=5) COVID-19 groups according to the National Early Warning Score (NEWS; mild <5, severe ≥5) evaluated on the day of blood sampling as previously published. The Scoring Algorithm of Molecular Subphenotypes (SAMS) was used to identify genomic risk profiles as previously described. SAMS Up and Down scores were calculated in each cohort using the product of two variables: the proportion of genes expected to be increased or decreased per subject (or single-cells) and their median, normalized gene expression levels. In this study, Up scores were calculated based on the expression of seven increased genes (PLBD1, TPST1, MCEMP1, IL1R2, HP, FLT3, S100A12) and Down scores based on the expression levels of 43 decreased genes (LCK, CAMK2D, NUP43, SLAMF7, LRRC39, ICOS, CD47, LBH, SH2D1A, CNOT6L, METTL8, ETS1, P2RY10, TRAT1, BTN3A1, LARP4, TC2N, GPR183, MORC4, STAT4, LPAR6, CPED1, DOCK10, ARHGAP5, HLA-DPA1, BIRC3, GPR174, CD28, UTRN, CD2, HLA-DPB1, ARL4C, BTN3A3, CXCR6, DYNC2LI1, BTN3A2, ITK, CD96, GBP4, SIPR1, NAP1L2, KLF12, IL7R) from a gene signature previously found to be predictive of IPF mortality. Two non-coding transcripts (SNHG1, C2orf27A) of the original gene signature were excluded because they were not consistently present across COVID-19 datasets. All of the samples with a 50-Gene, high-risk profile were classified as severe COVID-19 while 83.3% of samples with a low-risk profile were classified as mild COVID-19 (P=0.015) (FIG. 1a). 50-Gene, high-risk samples had significantly higher NEWS score (mean of 9.2 versus 1.8, P<0.001) and C-reactive protein (mean of 16.9 mg/dl versus 3.3 mg/dl, P=0.047) when compared to low-risk samples. Subjects in the low-risk profile had radiological evidence of pneumonia while subjects from the high-risk profile had evidence of multifocal pneumonia with ground glass opacities. Baseline 50-Gene risk profiles were also analyzed in a COVID-19 Validation cohort. (N=100 subjects, bulk peripheral blood leukocyte RNA-seq data, GEO Accession: GSE157103). SAMS cutoffs derived from the discovery cohort (Up score >0.41 and Down score <−0.41) distinguished high versus low-risk subjects in the validation cohort (FIG. 1b). High-risk subjects in the validation cohort were significantly older (64.8 versus 55 years, P=0.002), had higher APACHE-II severity score (22.5 versus 14.1, P=0.006), Charlson Comorbidity Index (4 versus 2.3, P<0.001), C-reactive protein (165.7 mg/l versus 101.3 mg/1, P=0.003), and Ferritin levels (1215.6 ng/ml versus 497 ng/ml, 0.002) when compared to low-risk subjects. High-risk subjects were more likely to have a prior history of myocardial infarction (16.9% versus 2.4%, P=0.02) and were more likely to receive convalescent plasma (32.2% versus 12.2%, P=0.02), and corticosteroid therapy (64.4% versus 14.6%, P<0.001). A 50-Gene, high-risk profile predicted ICU admission (Area Under the Curve—AUC: 0.77, 95% CI:0.686-0.844, P<0.001), mechanical ventilation (AUC: 0.75, 95% CI:0.67-0.827, P<0.001) and in-hospital mortality (AUC: 0.74, 95% CI:0.678-0.815, P<0.001) in the validation cohort. High-risk patients in the validation cohort spent more days on mechanical ventilation (21.9 versus 15.5 days, P<0.001) and had longer hospitalizations (21.1 versus 9 days, P<0.001) compared to low-risk patients. Only one patient in the 50-Gene, low-risk profile group died while 23 patients in the 50-Gene, high-risk profile group died during hospitalization (P=<0.001). All deceased patients in the validation cohort were in severe ARDS and on mechanical ventilation. Refractory respiratory failure was the cause of death in all the patients who died from COVID-19 in the validation cohort.

7-Gene Risk Profiles Derived from the 50-Gene Signature Predict Outcomes in COVID-19 Patients

To determine whether risk profiles derived from a combination of genes from the 50-Gene signature could be predictive of mechanical ventilation and mortality in hospitalized patients with COVID-19, whole blood was collected from a total of 140 patients recruited at USF/TGH. Quantitative RT-PCR was performed for all seven overexpressed genes in the gene signature (PLBD1, TPST1, MCEMP1, IL1R2, HP, FLT3 and S100A12). Transcript units [Tu=2 \hat{ }(10-dCt)], were calculated as previously described and SAMS Up scores were computed using the Tu of each one of these seven genes. SAMS Up scores were split by the median to determine COVID-19 high and low-risk subjects in this cohort (FIG. 4a). While 7-Gene, high-risk patients in this cohort did not have any significant differences in age, gender, CRP, procalcitonin or d-dimer levels when compared to low-risk patients, they did have increased levels of ferritin (2352 ng/ml versus 796 ng/ml, P=0.024), LDH (423 U/L versus 321 U/L, P<0.001) and troponin (0.65 versus 0.04 ng/ml). Only four patients died in the 7-Gene, low-risk profile group while 20 patients died in the 7-Gene, high-risk profile group during hospitalization (P=<0.001). To determine whether adding plasma levels of circulating cytokines could improve the outcome predictive accuracy of 7-Gene risk profiles in COVID-19, plasma levels of IL-1β, IL-6, IL-8, IL-10, IL-13, IFN-γ, CCL2, CCL18, Osteopontin (OPN), MMP1, and TGF-β were measured by Luminex in COVID-19 patients recruited at the USF/TGH cohort who also had 7-Gene expression measurements. Out of the tested cytokines, only IL-6, CCL2 and OPN were significantly predictive (P<0.05) of mortality in this cohort (N=132). None of the tested cytokines were significantly predictive of mechanical ventilation use. 7-Gene Up scores were found to be predictive of mechanical ventilation use (AUC:0.765, 95% CI:0.682 to 0.836, P<0.0001) and in-hospital mortality (AUC: 0.73, 95% CI:0.648-0.805, P<0.0001) in patients with COVID-19 in the USF/TGH cohort. The prediction of mechanical ventilation status using 7-Gene Up scores (AUC: 0.765) was superior to IL-6 (AUC: 0.59), CCL2 (AUC: 0.58) and OPN (AUC: 0.59). The prediction of in-hospital mortality using 7-Gene Up scores (AUC: 0.73) was superior to IL-6 (AUC: 0.64), CCL2 (AUC: 0.67) and OPN (AUC: 0.69) (FIG. 4c). To determine whether adding plasma levels of IL-6, CCL2 and OPN, measured by Luminex, could improve the mortality prediction accuracy of 50-Gene risk profiles, a logistic regression model was fit including 7-Gene Up scores, IL-6, CCL2 and OPN plasma levels. The model excluded IL-6 and demonstrated that adding CCL2 and OPN to 7-Gene Up scores increased the COVID-19 mortality prediction accuracy of the 7-Gene classifier with an AUC that went from 0.73 to 0.82. The results demonstrated that SAMS scores derived from components of the 50-Gene signature can be used to predict short-term outcomes in hospitalized patients with COVID-19. They also demonstrate the value of adding cytokines to improve the outcome prediction accuracy of the genomic classifier.

7-Gene Up Scores are Associated with Treatment Response in COVID-19 Patients

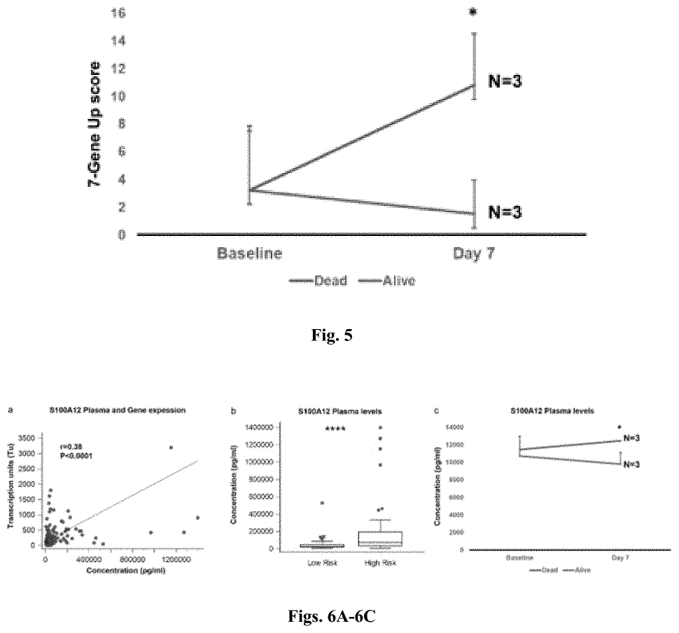

To determine whether temporal shifts in 50-Gene risk profiles are associated with response to COVID-19 treatments, whole blood RNA was extracted from COVID-19 hospitalized patients who survived (N=3) during hospital course or died (N=3) despite COVID-19 targeted therapies at USF/TGH. Gene expression of all seven overexpressed genes in the 50-Gene signature was measured using qRT-PCR. Gene Transcript Units (Tu) were used to measure 7-Gene Up scores. 7-Gene Up scores distinguished COVID-19 treated patients who survived from those who died during hospitalization (FIG. 5). The Up scores decreased from a median of 3.21 to 1.52 in the treated patients who survived while they increased from a median of 3.21 to 10.8 in the treated patients who died. The difference in 7-Gene Up scores between survivors and non survivors at the seventh day of hospitalization was statistically significant (P=0.022). These findings show that 50-Gene risk profiles can be used to monitor COVID-19 treatment response in the acute setting.

S100A12 Protein Levels in Plasma Correlate with their Gene Transcript Levels and are Associated with Treatment Response in COVID-19 Patients

S100A12 is one of 50 genes found to be associated with COVID-19 severity and poor outcomes when increased. To determine whether S100A12 plasma protein levels correlate with S100A12 transcript levels and with treatment response, plasma and whole blood gene expression levels were measured by qRT-PCR in patients with acute COVID-19 from the USF/TGH cohort at baseline (N=132 by Luminex) and during hospitalization (baseline and day 7) by ELISA in treated patients who survived (N=3) or died (N=3) during hospital course despite COVID-19 targeted therapies. Plasma levels of S100A12 measured by Luminex at baseline, were significantly correlated with gene transcript levels (r=0.38, P<0.0001) measured by qRT-PCR (FIG. 6a) and where significantly higher in COVID-19 patients with a 7-Gene, high-risk profile when compared to low-risk profile patients (P<0.0001) (FIG. 6b). It was also found that S100A12 levels decreased from a median of 10,717 pg/ml to 9,805 pg/ml in the treated patients who survived while they increased from a median of 3.21 to 10.8 in the treated patients who died (FIG. 6c). The difference in S100A12 plasma levels between survivors and non survivors at the seventh day of hospitalization was statistically significant (P=0.038). These results show that the S100A12 protein plasma levels and other translated proteins of the 50-Gene signature are used to monitor treatment response in hospitalized patients with COVID-19.

### Phenotypic Changes in Circulating Immune Cells are Responsible for 50-Gene Risk Profiles in COVID-19

To determine the cellular source of 50-Gene risk profiles in acute COVID-19, a publicly available COVID-19, scRNA-seq dataset was analyzed (N=7 subjects, N=155 single-cell measurements from PBMC, GEO accession: GSE150728 in a retrospective, multicenter cohort study. To determine cell types expressing either 50-Gene high or low-risk profiles in COVID-19, a cell-type-specific analysis was conducted using eight single-cell data measurements from seven subjects with COVID-19. The average expression levels are analyzed of each gene, for each cell type, producing 155 cell-type-specific expression profiles. SAMS high-risk cutoffs (Up score >0.41 and a Down score <−0.41), derived from the COVID-19 Discovery cohort (FIG. 1a) were used to distinguish cells with 50-Gene high or low-risk profiles. The estimated proportion (calculated by clustering) of specific cell types was compared between risk profiles. The cell type definition and classification has been previously described. SAMS high-risk cutoffs classified 47 cells with a high-risk profile and 108 cells with a low-risk profile (FIG. 3a). 50-Gene expressing cells with a high-risk profile mainly included CD14⁺ monocytes (16.7%), dendritic cells (16.7%) and neutrophils (16.7%), while 50-Gene expressing cells with a low-risk profile mainly included IgG producing plasmablasts (7.48%), mature CD4 T cells (7.48%) and naïve CD4 T cells (7.48%), CD8 mature T cells (7.48%), B cells (7.48%), Natural Killer cells (7.48%), proliferative lymphocytes (7.48%), gamma/delta T cells (6.54%) and Interferon stimulated CD4-T cells (5.41%) Cells with overlapping 50-Gene risk profiles included developing neutrophils, stem cells, eosinophils, myeloid cells, CD16 monocytes, platelets, plasmacytoid dendritic cells, IgA and IgM producing plasmablasts (FIG. 3b). These data show that performing combined scRNA-seq and cell-surface proteomics in PBMC are used to identify whether 50-Gene expressing cell proportions are predictive of COVID-19 mortality during acute COVID-19 and correlate with lung function post COVID-19 pneumonia.

3′ Gene Expression Library Construction from PBMC for Single-Cell RNA Sequencing

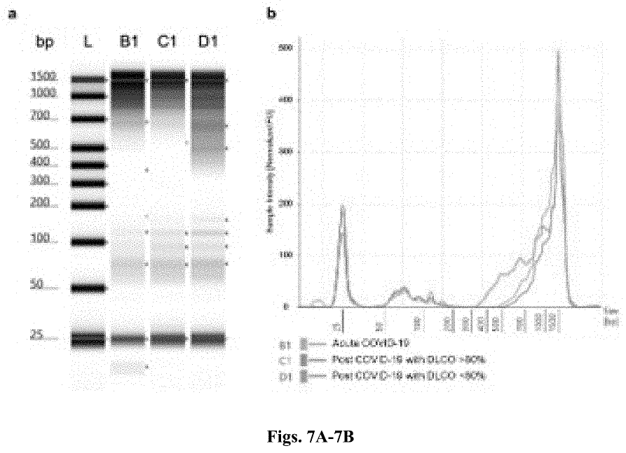

Cryopreserved PBMC were isolated from a patient with acute COVID-19 recruited at USF/TGH (Subject B1) and two patients with post COVID-19 pneumonia recruited at the USF/TGH-Center for Advanced Lung Disease (Subject C1 with DLCO % more than 80% and subject D1 with DLCO % less than 80%). PBMC were thawed, washed, and resuspended in PBS plus BSA at 0.04% for a final concentration of 1,000 cells per microliter using a Countess III automated cell counter. This was followed by the generation of Gel Beads-in-Emulsion (GEMs). GEMs contain barcoded, single cell 3′ gel beads that capture cell transcripts, a reverse transcription master mix containing the number of cells that is sequenced (approximately 5,000 per sample), and partitioning oil, all mixed on a Chromium Next GEM Chip G by the 10× Chromium controller. Following GEMs generation, gel beads were dissolved, releasing barcoded primers, and partitioned cells were lysed. The mix was immediately incubated for reverse transcription in a thermocycler, producing barcoded, full-length cDNA from cellular poly-adenylated mRNA. After incubation, silane magnetic beads were used to purify cDNA from the post-GEM-RT reaction mix containing biochemical reagents and primers. First, cDNA was subsequently amplified via polymerase chain reaction (PCR) to generate sufficient material for library construction and then purified. Next, amplified cDNA was enzymatically fragmented to optimize cDNA amplicon size. FIGS. 7a and 7b show cDNA quality control preceding library construction. FIG. 7a shows the migration profile and FIG. 7b demonstrate the electropherogram of the three cDNA samples (subjects B1, C1 and D1) obtained using an Agilent High Sensitivity D1000 ScreenTape assay kit to determine cDNA amplicon size. These results indicate that cDNA size in all three samples was above 1 kbp, which was optimal to pursue library construction. Additional sample index and primer sequences were incorporated on both ends of the barcoded cDNA via End Repair, A-tailing, adaptor ligation, and PCR, producing a Chromium single cell 3′, gene expression dual index library with paired-end constructs compatible with Illumina sequencing instruments. Library yield and quality were analyzed with an Agilent Tapestation 4150.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A method of treating a subject with severe COVID-19 or at risk of developing severe COVID-19, the method comprising:
a) obtaining a sample from the subject;
b) measuring expression level of at least two of the following genes from the subject: PLBD1, TPST1, MCEMP1, IL1R2, HP, FLT3 and S100A12;
c) determining a score for the measured expression level of the at least two genes;
d) using the scores of step c) to determine a severity signature profile;
e) determining that the severity signature profile of at least one of the two or more of the genes is greater than a gene-specific standard, such that expression of at least one of the two or more genes is increased; and
f) treating the subject for severe COVID-19 or treating the patient to prevent severe COVID-19.

2. The method of claim 1, wherein the subject has had known exposure to COVID-19.

3. The method of claim 1, wherein the subject is treated with one or more COVID-19 therapies.

4. The method of claim 3, wherein said one or more therapies include antivirals, corticosteroids, or monoclonal antibodies.

5. The method of claim 3, wherein the therapy comprises one or more of Remdesivir, Ritonavir-boosted nirmatrelvir, Bebtelovimab, Molnupiravir, Sotrovimab, dexamethasone, and other FDA approved therapies.

6. The method of claim 3, wherein the one or more COVID-19 therapies is given in a higher dose or for a longer time period than that given to a subject without severe COVID-19 or without a score that indicates that the subject could develop severe COVID-19.

7. The method of claim 1, wherein the subject is given a COVID-19 vaccine.

8. The method of claim 7, wherein the vaccine is given at a higher dose or more doses are given than would be given to a subject without COVID-19 or without severe COVID-19.

9. The method of claim 1, wherein the subject's oxygen level is measured at least once daily.

10. The method of claim 1, wherein the sample is a blood sample.

11. The method of claim 1, wherein said sample is a mucous sample.

12. The method of claim 1, wherein said sample is a nasal swab.

13. The method of claim 1, wherein at least three of PLBD1, TPST1, MCEMP1, IL1R2, HP, FLT3, and S100A12 are measured.

14. The method of claim 1, wherein at least four of PLBD1, TPST1, MCEMP1, IL1R2, HP, FLT3, and S100A12 are measured.

15. The method of claim 1, wherein at least five of PLBD1, TPST1, MCEMP1, IL1R2, HP, FLT3, and S100A12 are measured.

16. The method of claim 1, wherein at least six of PLBD1, TPST1, MCEMP1, IL1R2, HP, FLT3, and S100A12 are measured.

17. The method of claim 1, wherein each of PLBD1, TPST1, MCEMP1, IL1R2, HP, FLT3 and S100A12 are measured.

18. The method of claim 1, wherein at least 2, 3, 4, 5, 6, or all 7 of the genes have increased expression.

19. The method of claim 1, wherein Scoring Algorithm of Molecular Subphenotypes (SAMs) is used to calculate the score and the severity signature profile.

20. A method of treating a subject with severe COVID-19 or at risk of developing severe COVID-19, the method comprising:
a) obtaining a sample from the subject;
b) measuring expression level of at least one of the following genes from the subject: PLBD1, TPST1, MCEMP1, IL1R2, HP, FLT3 and S100A12;
c) measuring expression level of at least one of the following genes from the subject: LCK, CAMK2D, NUP43, SLAMF7, LRRC39, ICOS, CD47, LBH, SH2D1A, CNOT6L, METTL8, ETS1, C2orf27A, P2RY10, TRAT1, BTN3A1, LARP4, TC2N, GPR183, MORC4, STAT4, LPAR6, CPED1, DOCK10, ARHGAP5, HLA-DPA1, BIRC3, GPR174, CD28, UTRN, CD2, HLA-DPB1, ARL4C, BTN3A3, CXCR6, DYNC2LI1, BTN3A2, ITK, SNHG1, CD96, GBP4, S1PR1, NAP1L2, KLF12, IL7R);
d) determining a score for the measured gene expression level of the at least two genes from either step b), step c) or both;
e) using the score of step d) to obtain a severity signature profile;
f) determining that the severity signature profile is greater than a gene-specific standard;
g) treating the subject for severe COVID-19 or to prevent severe COVID-19.
